(19) United States
(12) Patent Application Publication
(10) Pub. No.: US 2016/0201107 A1
(43) Pub. Date: Jul. 14, 2016

(54) Salmonella and Listeria Assay Methods and Kits

(71) Applicant: Mississippi State University, Starkville, MS (US)

(72) Inventors: Tae Jo Kim, Starkville, MS (US); Juan L Silva, Starkville, MS (US)

(73) Assignee: MISSISSIPPI STATE UNIVERSITY, Starkville, MS (US)

(21) Appl. No.: 14/871,836

(22) Filed: Sep. 30, 2015

Related U.S. Application Data
(60) Provisional application No. 62/057,873, filed on Sep. 30, 2014.

(51) Int. Cl. C12Q 1/04; C12Q 1/10

(52) U.S. Cl. CPC C12Q 1/045; C12Q 1/04; C12Q 1/10

(57) ABSTRACT

Methods and kits for detection of bacteria, especially Salmonella spp. and Listeria spp., are provided using a unique combination of selective ingredients and two-phase culture (solid-phase culture gel and liquid-phase culture/enrichment broth) allows for high sensitivity and specificity of the kits for growth of Salmonella and Listeria in the detection methods and kits. The invention, through the detection mechanism accomplished by the novel formulation of selective ingredients and the two-phase culture, allows for real-time detection of a single cell of Salmonella and Listeria within 24±2 hours of introducing a target sample to the Salmonella and Listeria detection kits. The detection methods and kits for Salmonella may optionally further comprise a confirmation step that can increase sensitivity and accuracy for this pathogenic bacteria.

### CROSS-REFERENCES TO RELATED APPLICATIONS

This application claims the benefit of U.S. Provisional Application No. 62/057,873 by Kim and Silva, filed Sep. 30, 2014, which is incorporated herein by reference in its entirety.

### STATEMENT REGARDING FEDERALLY SPONSORED RESEARCH OR DEVELOPMENT

This invention was made with Government support under 58-6402-2-729 awarded by the Agricultural Research Service, USDA, and 0214027 and 0233884 awarded by the National Institute of Food and Agriculture (NIFA), USDA. The Government has certain rights in the invention.

### FIELD OF THE INVENTION

The present invention relates to the detection and quantification of the presence of certain bacteria, including Salmonella and Listeria, in the food supply, livestock feed supply, water, non-food materials, clinical samples (e.g., human patient), animal samples (e.g., veterinarian or meat processing facilities), and environmental samples in laboratory, field, or industrial settings with a high sensitivity.

### BACKGROUND OF THE INVENTION

The use of kits to detect bacteria (e.g., Vibrio spp. sometimes referred to herein as Vibrio for brevity) in the environment, industrial settings, or on food is known. U.S. Pat. No. 4,308,347 discloses a container device to detect pathogenic microorganisms in a fluid sample. The use of color generating chemicals was conceived as a mechanism to indicate the presence of pathogenic organisms with the intended result of a reduced need to use additional analysis equipment. U.S. Pat. No. 5,786,167 discloses a method to distinguish pathogen species such as those of Salmonella bacteria (sometimes referred to herein as Salmonella for brevity) by plating the sample on a solid medium of melibiose, mannitol, sorbitol, a pH indicator, and chromogenic substrate to reveal the presence of particular bacteria. If the test is positive, the bacteria is then cultivated and tested for additional indication for the presence of target pathogen specifically. U.S. Pat. No. 5,843,699 discloses a method of recognition and classification from a pre-enriched media that discourages the growth of non-target organisms while encouraging the growth of target microorganisms. The sample is then incubated and subjected to biochemical testing and analysis for results. To increase sensitivity, the prior art also discloses mechanisms to reduce the growth or generation of other bacteria within test samples, such as U.S. Pat. No. 5,208,150, which incorporates sodium salt to suppress the growth of competing organisms.

U.S. Pat. No. 6,136,554 discloses a method to detect E. coli, Salmonella, or a mixture of both by inoculating a nutrient medium into a sample and reviewing the color of bacterial colonies grown in the sample. If the result is negative, the process is repeated using different biochemical tests for different bacteria.

Salmonella and Listeria are leading causes of food-borne illnesses in the United States and throughout the world. More analytical resources are needed to monitor the food supply, agricultural materials, animal feed, water samples, and other environmental samples for these and other bacterial contaminants (e.g., surge resources in the event of an outbreak and mitigation resources for sample monitoring). The prior art does not disclose a cultural-based mechanism or kit to detect and quantify the presence and amount of pathogenic Salmonella and Listeria at a high-sensitivity rate, that is more accurate, that significantly reduces the occurrence of Type-I or Type-II errors such as false-positives and false-negatives, that can be performed in a significantly reduced time period, that can be accurate and reproducible without the use of additional laboratory machinery or equipment, and that can be performed through a single-tube methodology.

Therefore, a need in the art for Salmonella, Listeria, and other bacterial pathogen assay kits with the previously mentioned attributes is evident.

### SUMMARY OF THE INVENTION

The present invention provides a series of detection methods and novel bacterial assay kits useful for detecting and quantifying the presence of Salmonella and Listeria in the food supply, livestock feed supply, water, non-food materials, clinical samples (e.g., human patient), animal samples (e.g., veterinarian or meat processing facilities), and environmental samples in laboratory, field, or industrial settings with a high sensitivity. The present invention provides a simple, inexpensive mechanism to detect Salmonella spp., Listeria welshimeri, Listeria innocua, Listeria ivanovii, Listeria grayi, Listeria seeligeri and Listeria monocytogenes (which may be referred to herein as Salmonella and Listeria for brevity) in food, feed, and other materials, and environmental samples with a very high sensitivity while also preventing the occurrence of false-positives, false-negatives, or other statistical errors. A novel combination of selective ingredients and two-phase culture (solid-phase culture gel and liquid-phase culture/enrichment broth) allows for high sensitivity and specificity of the kits for growth of Salmonella and Listeria in the detection kits. The present invention, through the detection mechanism accomplished by the novel formulation of selective ingredients and the two-phase culture, allows for real-time detection of a single cell of Salmonella and Listeria within 24±2 hours of introducing a target sample to the Salmonella and Listeria detection kits. An additional confirmatory step that is optional for Salmonella assay methods and kits may then include a second 24±2 hours period for confirmation detection results. An additional benefit of the present invention is that it does not require the use of additional machinery/equipment or highly skilled personnel to detect the presence of Salmonella and Listeria in a test sample. The present invention also does not require additional machinery or equipment to read or interpret the results of the detection mechanism, unlike the current state of technology.

It is the object of the present invention to provide a series of methods and kits to detect Salmonella and Listeria in the food supply, livestock feed supply, water, non-food materials, clinical samples (e.g., human patient), animal samples (e.g., veterinarian or meat processing facilities), and environmental samples in laboratory, field, or industrial settings with a high sensitivity, through the use of single-tube Salmonella and Listeria assay kits. It is an additional object of the present invention to describe a methodology for detecting Salmonella and Listeria in food, material, water and environment samples in a Salmonella and Listeria assay kit that reduces the opportunity for Type-I or Type-II errors while also limiting the time and additional resources traditionally required for detecting the presence of Salmonella and Listeria.

In one aspect, the present invention relies on a unique combination of selective media, chemicals, temperature, and motility activity of live Listeria cells that promote the facultative anaerobic growth of Listeria while also inhibiting the growth of other microorganisms. For example, lithium chloride, acriflavine, cefotetan, colistin sulfate, cycloheximide and fosfomycin in Oxford medium are examples of chemicals that are known in the industry to increase selectivity Listeria at the exclusion of other microorganisms. Utilizing the combination of these selective media, chemicals and two-phase culture technology, the Listeria kits can quickly increase selectivity of Listeria in a test sample; this enhanced selectivity can then be evaluated and evidenced through the two-phase culture by altering the characteristics of signal indicating chemicals, such as esculin and ferric ammonium citrate as indicators which, in turn, will provide visible evidence of the presence of Listeria.

In another aspect, the present invention relies on a unique combination of biomarker expression, selective/non-selective media, antibiotics, chemicals, growth temperature, and motility activity of viable Salmonella cells that promote the facultative anaerobic growth of Salmonella while also inhibiting the growth of other microorganisms. For example, malachite green oxalate in Rappaport-Vassiliadis R10 broth (RVB) and brilliant green (BG) and novobiocin in brilliant green agar are examples of chemicals that are known in the industry to increase selectivity of Salmonella by the exclusion of other microorganisms. Utilizing the combination of these selective media and chemicals and two-phase culture technology, the Salmonella kits can quickly increase selectivity of Salmonella in a test sample; this enhanced selectivity can then be evaluated and evidenced through the two-phase culture by altering the characteristics of a signal indicating medium, such as phenol red as an indicator which, in turn, will provide visible evidence of the presence of Salmonella.

### BRIEF DESCRIPTION OF THE DRAWINGS

Further advantages of the invention will become apparent by reference to the detailed description of preferred embodiments when considered in conjunction with the drawings:

FIG. 1 depicts four Listeria vessels containing samples known to be negative for Listeria (left side of test tube rack), and all are negative for Listeria presence in an exemplary embodiment method of the present invention, which can be seen with the yellow (remaining) color of the bottom agar media. Also shown is a fifth Listeria vessel containing a sample known to be positive for Listeria (far right), and it is positive for Listeria presence in an exemplary embodiment method of the present invention, which can be seen with the black (changed) color of the bottom agar media.

FIGS. 2A, 2B, 2C, and 2D depict vessels I containing samples known to be positive for Salmonella, and all are positive for Salmonella presence in an exemplary embodiment method of the present invention, which can be seen with the orange (changing) color of the bottom agar media in the vessels I. FIG. 2E depicts a vessel I containing samples known to be negative for Salmonella, and is negative for Salmonella presence in an exemplary embodiment method of the present invention, which can be seen with the green (remaining) color of the bottom agar media in the vessels I. FIG. 2F depicts vessels II labeled in the figures as vessels a, b, c, and d. Vessels a, b, and c all contain known positive Salmonella samples, and each shows positive for Salmonella presence in an exemplary embodiment method of the present invention, which can be seen with the yellow and/or orange (changing) color of the bottom agar media in the vessels II. Vessel d, however, contains a known negative Salmonella sample, and this vessel II is negative for Salmonella presence in an exemplary embodiment method of the present invention, which can be seen with the green to light green (remaining) color of the bottom agar media in the vessel II.

FIGS. 3A-B depict the confirmatory Salmonella vessels III in in an exemplary embodiment method of the present invention. FIG. 3A shows triplicate vessels III after inoculation from liquid-phase media following either vessel I or vessel II culturing shown in FIGS. 2A-2F. FIG. 3B shows the vessels III after incubation for 24 hours at 42° C. As shown in FIG. 3B, triplicate vessels III marked “1” and “2” contain inoculant from positive Salmonella samples from either a vessel I sealable bag or a vessel II sample, and each shows positive for Salmonella presence, which can be seen with the orange (changing) color of the bottom agar media and yellowing of the liquid-phase media in the vessels III. The triplicate vessels III marked “3” (one tube shown at left of test tube rack), however, contains inoculant from the negative Salmonella sample from a vessel I or vessel II test, and this vessel III is negative for Salmonella presence, which can be seen with the green to light green (remaining) color of the bottom agar media and liquid-phase media in the vessel III.

### DETAILED DESCRIPTION

The following detailed description is presented to enable any person skilled in the art to make and use the invention. For purposes of explanation, specific details are set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments described, but is to be accorded the widest possible scope consistent with the principles and features disclosed herein.

The present invention provides a series of detection methods and novel bacterial assay kits useful for detecting and quantifying the presence of Salmonella and Listeria and other bacteria in the food supply, livestock feed supply, water, non-food materials, clinical samples (e.g., human patient), animal samples (e.g., veterinarian or meat processing facilities), and environmental samples in laboratory, field, or industrial settings with a high sensitivity. The present invention provides a simple, inexpensive mechanism to detect Salmonella spp., Listeria welshimeri, Listeria innocua, Listeria ivanovii, Listeria grayi, and Listeria seeligeri and Listeria monocytogenes (which may be referred to herein as Salmonella and Listeria for brevity) in the food supply, livestock feed supply, water, non-food materials, clinical samples (e.g., human patient), animal samples (e.g., veterinarian or meat processing facilities), and environmental samples in laboratory, field, or industrial settings with a high sensitivity while also preventing the occurrence of false-positives, false-negatives, or other statistical errors. A novel combination of selective ingredients and two-phase culture (solid-phase culture gel and liquid-phase culture/enrichment broth) allows for high sensitivity and specificity of the kits for growth of Salmonella and Listeria in the detection kits. The present invention, through the detection mechanism accomplished by the novel formulation of selective ingredients and the two-phase culture, allows for real-time detection of a single cell of Salmonella and Listeria within 24±2 hours of introducing a target sample to the Salmonella and Listeria detection kits. An additional confirmatory step that is optional for Salmonella assay methods and kits may then include a second 24±2 hours period for confirmation detection results. An additional benefit of the present invention is that it does not require the use of additional machinery/equipment, such as expensive real-time PCR equipment and consumable reagents, or highly skilled personnel to detect the presence of Salmonella and Listeria in a test sample. The present invention also does not require additional machinery or equipment to read or interpret the results of the detection mechanism, unlike the current state of technology.

The disclosed present invention provides methods and kits to detect Salmonella and Listeria in food, feed, other materials, water, and environment samples in laboratory, field, and industrial settings, through the use of single-tube Salmonella and Listeria assay kits. The disclosed present invention also provides a methodology for detecting Salmonella and Listeria in the food supply, livestock feed supply, water, non-food materials, clinical samples (e.g., human patient), animal samples (e.g., veterinarian or meat processing facilities), and environmental samples in laboratory, field, or industrial settings with a high sensitivity in a Salmonella and Listeria assay kit that reduces the opportunity for Type-I or Type-II errors while also limiting the time and additional resources traditionally required for detecting the presence of Salmonella and Listeria, such as culture methods that require several culturing steps to obtain culture identification, ELISA methods, or molecular methods, including DNA probes and PCR. In preferred embodiments, esculin hydrolysis in the solid-phase media by Listeria increases sensitivity of the Listeria assay kit (reducing Type II errors) to detect Listeria spp. In other preferred embodiments, buffered peptone water as a liquid-phase media increases the sensitivity of the Salmonella assay vessels (reducing Type II errors) to detect Salmonella spp. In further preferred embodiments, RVS with high osmotic pressure, carboxylase activity at low pH, and malachite green as a liquid-phase media increases the sensitivity of the Salmonella assay vessels (reducing Type II errors) to detect Salmonella spp. To reduce Type-I errors for: (a) nalidixic acid, acryflavin HCl in Listeria enrichment broth and antibiotics known in the field to suppress non-Listeria bacteria in Oxford media inhibit non-Listeria bacteria; and (b) brilliant green, magnesium chloride, and high temperature incubation (42° C.) inhibit the growth of non-Salmonella microorganisms.

In some embodiments, the present invention relies on a unique combination of selective media, chemicals, temperature, and motility activity of live Listeria cells that promote the facultative anaerobic growth of Listeria while also inhibiting the growth of other microorganisms. For example, lithium chloride, acriflavine, cefotetan, colistin sulfate, cycloheximide and fosfomycin in Oxford medium are examples of chemicals that are known in the industry to increase selectivity Listeria at the exclusion of other microorganisms. Utilizing the combination of these selective media, chemicals and two-phase culture technology, the Listeria kits can quickly increase selectivity of Listeria in a test sample; this enhanced selectivity can then be evaluated and evidenced through the two-phase culture by altering the characteristics of signal indicating chemical(s), such as esculin and ferric ammonium citrate as indicators which, in turn, will provide visible evidence of the presence of Listeria.

In other embodiments, the present invention relies on a unique combination of biomarker expression, selective/non-selective media, antibiotics, chemicals, growth temperature, and motility activity of viable Salmonella cells that promote the facultative anaerobic growth of Salmonella while also inhibiting the growth of other microorganisms. For example, malachite green oxalate in Rappaport-Vassiliadis R10 broth (RVB) and brilliant green (BG) and novobiocin in brilliant green agar are examples of chemicals that are known in the industry to increase selectivity of Salmonella by the exclusion of other microorganisms. Utilizing the combination of these selective media and chemicals and two-phase culture technology, the Salmonella kits can quickly increase selectivity of Salmonella in a test sample; this enhanced selectivity can then be evaluated and evidenced through the two-phase culture by altering the characteristics of a signal indicating chemical(s), such as phenol red as an indicator which, in turn, will provide visible evidence of the presence of Salmonella.

Listeria

The following provides detailed descriptions of the methods and kits to detect Listeria ssp. (“Listeria”) in the food supply, livestock feed supply, water, non-food materials, clinical samples (e.g., human patient), animal samples (e.g., veterinarian or meat processing facilities), and environmental samples in laboratory, field, or industrial settings with a high sensitivity, through the use of Listeria assay kits. In preferred embodiments, the detection of pathogenic Listeria is conducted by utilizing the disclosed the methods and kits.

A single-tube two-phase (biphasic) culture assay system is provided with sufficient specificity for the identification and detection of Listeria in complex matrices, such as food, agricultural materials, or environmental samples, which may also contain interfering bacteria that mimic these bacteria. The disclosed system combines broth enrichment and selective isolation/identification into a single-tube assay methodology that can provide accurate screening results in about 24 hours from inoculation. The state of the art utilizes advanced molecular methods, such as real-time PCR, but these are very expensive and laborious (require specially-trained personnel and expensive equipment and consumable reagents). The low cost and simplicity of the disclosed elegant solution allows for rapid implementation and maintained monitoring of the food, water, environmental, and other sources of Listeria contamination or exposure. The disclosed test kit media can be prepared and stored (at room temperature or chilled) for up to six months, which provides for a longer shelf life than some paper indicator strip kits.

Sample Preparation and Optional Enrichment—In some embodiments, a sample (e.g., environmental sample swabs) may be directly incubated in the Listeria detection vessel that is pre-filled with the top and bottom gels and liquid-phase culture/enrichment broth (preparation of growth/selection and detection media discussed below). In other embodiments, a sample (e.g., food and agricultural materials) may go through an optional enrichment step performed to enhance the detection sensitivity of the test. For example, a food sample (25 g) is placed in a sterile sealable plastic bag (e.g., ZIPLOCK® brand bag or STOMACHER® brand bag) or other culture container and mixed with appropriate amount of sterile Listeria liquid-phase culture/enrichment broth at room temperature or pre-warmed temperature (35-37° C.). The enrichment step can be incubated overnight or up to 24 hours before placing an appropriate volume of the Listeria liquid-phase culture/enrichment broth in one or more single-tube Listeria kit vessel with solid-phase top and bottom gels. An inoculated single-tube Listeria kit vessel (with or without the optional enrichment step) is then incubated at 35-37° C. for 24 hours before reading for detection of Listeria.

Formulation of Listeria Liquid-Phase Culture/Enrichment Broth—In a preferred formulation, Listeria liquid-phase culture/enrichment broth is prepared with 3% tryptic soy broth, 1.5% sodium chloride, and 0.0067% acriflavine HCl. In alternative formulation embodiments, the tryptic soy broth, sodium chloride, and acriflavine HCl can each be used at concentrations between 0.0001-10%. In further alternative formulation embodiments, the tryptic soy broth can be replaced with another non-selective nutrient broth such as: buffered peptone water, peptone water, nutrient broth, lactose broth, Mueller-Hinton broth, brain heart infusion broth, or their modified formulas known in the art. Commercially available Listeria enrichment broth such as UVM modified Listeria enrichment broth or Listeria enrichment broth can be replaced as a liquid-phase culture broth. Compounds that enhance the recovery of stressed or injured cells, such as pancreatic digest of casein, proteose peptone #3, beef extract, yeast extract sodium chloride, disodium phosphate, monopotassium phosphate, esculin in Listeria enrichment broth, may also be included in this phase to optimize the sensitivity and recovery. As indicated above, the Listeria liquid-phase culture/enrichment broth can be inoculated in an optional enrichment step or inoculated directly in a Listeria kit vessel. The Listeria liquid-phase culture/enrichment broth is formulated to enhance Listeria growth while suppressing (e.g., via nalidixic acid and acriflavin HCl) other bacteria and microflora in a given sample.

Formulation of Solid-Phase Culture Bottom Gel—The solid-phase will suppress growth of background microflora by incorporation of antimicrobial compounds and differential aspects will be achieved based on the unique biochemical utilization patterns of Listeria with indicator compounds and dyes. In some embodiments, the solid-phase culture bottom gel can be prepared with commercially available Oxford medium base or equivalent components (0.89% pancreatic digest of casein, 0.44% proteose peptone No. 3, 0.44% yeast extract, 0.27% beef heart, infusion from 500 g, 0.09% starch, 0.44% sodium chloride, 0.01% esculin, 0.05% ferric ammonium citrate, 0.15% lithium chloride and 0.153% agar), and supplemented with 0.1-0.5% esculin. Adding 0.8% commercially available Oxford Listeria supplement is optional. Oxford Listeria supplement contains acriflavine (5 mg), cefotetan (2 mg), colistin sulfate (20 mg), cycloheximide (400 mg), and fosfomycin (10 mg). Oxford Listeria supplement formulations containing these or equivalent components can be secured from a variety of commercial vendors. In some embodiments, alternative formulations may contain esculin and/or ferric ammonium citrate between 0.01-5% in final concentration. In further embodiments, alternative formulations may contain sodium chloride, lithium chloride, magnesium chloride, calcium chloride, agar, and/or starch between 0-15% in final concentration. In still further embodiments, the protein and amino acid sources of pancreatic digest of casein, proteose peptone no. 3, yeast extract, beef heart infusion may alternatively be individually or wholesale replaced with other proteins, peptides or amino acids sources for formulations including proteose peptone #1 and #2, gelatin, milk protein (casein), or other animal and plant proteins. In preferred embodiments, the agar component is replaced with another gelling agent, such as pectin or gelatin with calcium chloride or magnesium chloride. Depending on the desired matrix properties of the solid-phase gel, these can each be used in concentrations of from 0.01% to 10%. In a more preferred embodiment, low methoxyl pectin with calcium chloride or magnesium chloride from 0.01% to 10% is used in place of agar (see below for examples of pectin solid-phase media for Listeria detection and quantification). The use of pectin in place of agar saves time and energy in the preparation of the solid-phase media and the pectin kit components are easier to prepare at room temperature.

Formulation of Solid-Phase Culture Top Gel—In some embodiments, the solid-phase culture top gel can be prepared with 1% peptone and 1% agar. In some embodiments, alternative formulations may contain peptone and agar between 0.01-10% in final concentration. In further embodiments, the protein and amino acid source of peptone may alternatively be replaced with other proteins, peptides or amino acids sources for formulations including proteose peptone, gelatin, milk protein (casein), or other animal and plant proteins. In preferred embodiments, the agar component is replaced with another gelling agent, such as pectin or gelatin with calcium chloride or magnesium chloride. Depending on the desired matrix properties of the solid-phase gel, these can each be used in concentrations of from 0.01% to 10%. In a more preferred embodiment, low methoxyl pectin with calcium chloride or magnesium chloride from 0.01% to 10% is used in place of agar (see below for examples of pectin solid-phase media for Listeria detection and quantification). The use of pectin in place of agar saves time and energy in the preparation of the solid-phase media and the pectin kit components are easier to prepare at room temperature.

Preparation of Listeria Agar Kit Vessel with Two Solid-Phase Culture Gels (Bottom and Top) and Liquid-Phase Culture/Enrichment Broth (a Preferred Embodiment)

1. As a bottom solid-phase, a formulation of the solid-phase culture bottom gel agar media (described above) is autoclaved at 121° C. and 15 psi for 15 min.

2. Then, the autoclaved bottom gel agar media is cooled to approximately 50° C.

3. In laboratory studies, 4 mL of bottom gel agar media is placed in a sterilized glass tube (18 mm O.D.×38 mm length) and cooled at room temperature until solidified.

4. As a top solid-phase, a formulation of the solid-phase culture top gel agar media (described above) is autoclaved at 121° C. and 15 psi for 15 min.

5. Then, the autoclaved top gel agar media is cooled to approximately 50° C.

6. The cooled top gel agar media (2 mL) is placed on pre-filled bottom gel agar media and cooled at room temperature until solidified.

7. As a liquid phase, a formulation of the sterile liquid-phase culture/enrichment broth (described above) is autoclaved at 121° C. and 15 psi for 15 min, then allowed to cool to room temperature.

8. The cooled sterile liquid-phase culture/enrichment broth (10-20 mL) is used as an enrichment broth in a tube filled with bottom and top gel agar media. As discussed above, the sterile liquid-phase culture/enrichment broth can be included in an optional enrichment culture step prior to adding this media the Listeria kit vessel(s). Thus, the sterile liquid-phase culture/enrichment broth can be added to the Listeria kit vessel(s) prior to inoculation or inoculated post-optional enrichment culture step liquid-phase culture/enrichment broth can be added to the Listeria kit vessel(s) here.

9. The Listeria agar assay kit vessel is now prepared for use. The individual assay formulations (including top gel agar media and bottom gel agar media and sterile liquid-phase culture/enrichment broth) can be stored separately under standard operating procedures and conditions; and then combined before use as provided above.

As discussed above, alternative formulations of the media components can be used in other embodiments (i.e., other agar or solid-phase/gelling agent mixtures, selective media, chemicals, volume, and concentration, etc.) of the present invention.

Preparation of Listeria Pectin Kit Vessel with Two Solid-Phase Culture Gels (Bottom and Top) and Liquid-Phase Culture/Enrichment Broth (a Preferred Embodiment)

1. As a bottom solid-phase, a formulation of the solid-phase culture bottom soft gel agar (0.3%) media (described above) is autoclaved at 121° C. and 15 psi for 15 min. The soft gel agar media can be 0.1% to 0.5% agar.

2. Then, the autoclaved bottom gel pectin media is cooled at room temperature.

3. In laboratory studies, 4 mL of bottom gel agar media is placed in a sterilized glass tube (18 mm O.D.×38 mm length).

4. As a top solid-phase, a formulation of the solid-phase culture top gel pectin (1%) media (described above) is autoclaved at 121° C. and 15 psi for 15 min.

5. Then, the autoclaved top gel pectin media is cooled at room temperature.

6. 2% calcium chloride (50-500 μL) is immediately added to the top pectin media to gel the pectin.

7. The cooled top gel pectin media (2 mL) is placed on pre-filled bottom gel pectin media and cooled at room temperature until solidified.

8. As a liquid phase, a formulation of the sterile liquid-phase culture/enrichment broth (described above) is autoclaved at 121° C. and 15 psi for 15 min, then allowed to cool to room temperature.

9. The cooled sterile liquid-phase culture/enrichment broth (10-20 mL) is used as an enrichment broth in a tube filled with bottom and top gel pectin media. As discussed above, the sterile liquid-phase culture/enrichment broth can be included in an optional enrichment culture step prior to adding this media the Listeria kit vessel(s). Thus, the sterile liquid-phase culture/enrichment broth can be added to the Listeria kit vessel(s) prior to inoculation by sample.

10. The Listeria pectin assay kit vessel is now prepared for use. The individual assay formulations (including top gel pectin media and bottom soft gel media and sterile liquid-phase culture/enrichment broth) can be stored separately under standard operating procedures and conditions; and then combined before use as provided above.

The alternative formulations of Listeria media described above for the agar kits may also be used to supplement or modify the pectin kits.

Listeria Assay Kits

The present invention relates to novel Listeria assay kits comprised of a novel combination of selective ingredients that can be used to detect and quantify the population of Listeria, including pathogenic Listeria in preferred embodiments, present in a test sample. The Listeria assay kits include, but are not limited to, the following: instructions for storage, use, and detection/quantification; assay block/vessel(s); selective and/or non-selective pre-enrichment liquid media; selective solid or semi-solid media; selective liquid media; and color chart to read sample.

The Listeria assay kits include a singular or a plurality of vessels (which may include test tubes, culture blocks, microculture plates, or other vessel form of appropriate size based on sample screening size or through-put requirements) whereby each vessel contains biochemicals of the detection mechanism in a stable matrix coupled with a growth medium in the same test vessel also in a stable matrix. Preferably, the Listeria assay kit also includes a specific set of instructions for storage, use, and detection. The Listeria assay kit may also contain a color chart specific to the signal indicating medium (biochemicals of the detection mechanism) provided. The Listeria assay kit may be sold on an individual or bulk basis and may be incorporated to include components necessary to perform the steps or processes common to the process of obtaining a viable sample from the environment, industrial setting, water, food sample, or other sample source.

Listeria Assay Kit Methods

Step 1 (Inoculation/Incubation): Test samples are taken in accordance with normal and well-known laboratory and industrial setting procedures. Test samples of an appropriate dilution(s) are inoculated by mixing sample(s) with Listeria enrichment broth (liquid-phase culture media). Swabbed samples using cotton swab or other instrument can be directly inoculated in the kit filled with Listeria enrichment broth (liquid-phase culture media) in the appropriately prepared Listeria assay kit and incubated at 30-37° C. for a period of about 24 hours. For quantification, replicates of serial dilutions are inoculated and incubated in Step 1 following well-known procedures for most probable number analysis (see, e.g., Scott Sutton, The Most Probable Number Method and Its Uses in Enumeration, Qualification, and Validation, J. Valid. Tech. vol. 16, no. 3 (Summer 2010), which is hereby incorporated by reference in its entirety). For example, dilutions can be log base 10, log base 2, or other serial dilution.

Step 2 (Reading): After about 24 hours of incubation, a positive result for Listeria in a Listeria assay kit sample shows black color on the bottom of vessel (bottom gel media) while negative samples stay yellow/amber color on the bottom (based on the biochemical indicators included in the preferred embodiments described above).

Salmonella

The following provides detailed descriptions of the methods and kits to detect Salmonella spp. (“Salmonella”) in the food supply, livestock feed supply, water, non-food materials, clinical samples (e.g., human patient), animal samples (e.g., veterinarian or meat processing facilities), and environmental samples in laboratory, field, or industrial settings with a high sensitivity, through the use of Salmonella assay kits. In preferred embodiments, the detection of Salmonella is conducted by utilizing the disclosed methods and kits.

A single-tube two-phase (biphasic) culture assay system is provided with sufficient specificity for the identification and detection of Salmonella in complex matrices, such as food, agricultural materials, or environmental samples, which may also contain interfering bacteria that mimic these bacteria. The disclosed system combines broth enrichment and selective isolation/identification into a single-tube assay methodology that can provide accurate screening results in about 24±2 hours from inoculation. An optional confirmatory step can be added that will result in final detection results within 24±2 hours from inoculation of the confirmation test. The state of the art utilizes advanced molecular methods, such as real-time PCR, but these are very expensive and laborious (require specially-trained personnel and expensive equipment and consumable reagents). The low cost and simplicity of the disclosed elegant solution allows for rapid implementation and maintained monitoring of the food, feed, water, environmental, and other sources of Salmonella contamination or exposure. The disclosed test kit media can be prepared and stored (at room temperature or chilled) for up to six months, which provides for a longer shelf life than some paper indicator strip kits.

Sample Preparation and Optional Enrichment—In some embodiments, a sample (e.g., environmental sample swabs) may be directly incubated in the Salmonella detection vessel (Salmonella Kit Vessel I or II) that is pre-filled with the bottom gel and liquid-phase culture broth and incubated overnight or up to 24 hours at 41° C.±1° C. (preparation of growth/selection and detection media for Salmonella Kit Vessels I-III is discussed below). In other embodiments, a sample (e.g., food, feed, or other materials), an optional enrichment step may be performed to enhance the detection sensitivity of the test. For example, a food sample (25-325 g) is placed in a sterile sealable plastic bag (e.g., ZIPLOCK® brand bag or STOMACHER® brand bag) or other culture container and mixed with an appropriate amount of sterile liquid-phase culture/enrichment broth (sterile buffered peptone water) at room temperature or pre-warmed temperature (37-42° C.). The enrichment step can be incubated overnight or up to 24 hours at 41° C.±1° C. with three (3) to five (5) single-tube vessels (Salmonella Kit Vessel I) added directly to the plastic bag/container. Alternatively, an appropriate volume of the inoculated liquid-phase culture/enrichment broth can then be placed in one or more single-tube vessels (Salmonella Kit Vessel II) with solid-phase top and bottom gels and incubated overnight or up to 24 hours at 41° C.±1° C. After incubation, the single-tube vessels (Salmonella Kit Vessel I or Salmonella Kit Vessel II) are read for detection of Salmonella and further processed as indicated.

Formulation of Salmonella Liquid-Phase Culture/Enrichment Broth—In a preferred formulation, Salmonella liquid-phase culture/enrichment broth is prepared as sterile buffered peptone water, which contains 1% peptone, 0.5% sodium chloride, 0.35% disodium phosphate, 0.15% potassium dihydrogen phosphate, pH 7.2±0.2 or an equivalent formula or commercially available product. In alternative formulation embodiments, the buffered peptone water can be replaced with other non-selective nutrient broths such as tryptic soy broth, peptone water, nutrient broth, lactose broth, Mueller-Hinton broth, brain heart infusion broth or their modified formula, or their modified or equivalent formulas known in the art. Compounds (peptone, sodium chloride, disodium phosphate, potassium dihydrogen phosphate in buffered peptone water as a liquid phase in the kit) that enhance the recovery of stressed or injured cells may also be included in this phase to optimize the sensitivity and recovery. As indicated above, the Salmonella liquid-phase culture/enrichment broth can be inoculated in an optional enrichment step or inoculated directly in a Salmonella kit vessel. Preferably, the Salmonella liquid-phase culture/enrichment broth for initial test samples (Vessels I & II) is formulated to be non-selective of enhancing Salmonella growth in a given sample.

For Salmonella confirmation (Vessel III) inoculation/incubation test samples, the Salmonella Liquid-Phase Culture/Enrichment Broth is replaced with Rappaport-Vassiliadis Salmonella (RVS) Broth, RV R10 broth, or equivalent alternatives with malachite green oxalate or Brilliant Green (0.1-100 ppm) that are well-known in the field. These media and their formulas are well known in the art for selective enrichment of Salmonella over other microorganisms in culture, especially E. coli, and many are commercially available from multiple vendor sources.

Formulation of Solid-Phase Culture Gel—The solid-phase will suppress growth of background microflora by incorporation of antimicrobial compounds and differential aspects will be achieved based on the unique biochemical utilization patterns of Salmonella with indicator compounds and dyes. In some embodiments, the solid-phase culture gel can be prepared with 0.5% NaCl, 1% peptone, 2% agar and 1% magnesium chloride, 12.5 ppm Brilliant Green, 0.8% phenol red (1% phenol red, sodium salt stock in sterilized distilled water). In some embodiments, alternative formulations with Brilliant Green may be modified in concentration or replaced with malachite green oxalate, crystal violet, other antibiotics (e.g., novobiocin) known in the art, or their combination, where the concentration of Brilliant Green and other antibiotics could be between 0.01-1,000 ppm in final concentration. In further embodiments, alternative formulations may contain sodium chloride, peptone (or other protein/amino acid sources), magnesium chloride, calcium chloride, and agar at between 0-15% in final concentration. In still further embodiments, the peptone may alternatively be replaced with other proteins, peptides or amino acids sources for formulations including proteose peptone, gelatin, milk protein (casein), or other animal and plant proteins. In still yet further embodiments, the phenol red can be replaced with other pH indicators or dye compounds. In preferred embodiments, the agar component is replaced with another gelling agent, such as pectin or gelatin with calcium chloride or magnesium chloride. Depending on the desired matrix properties of the solid-phase gel, these can each be used in concentrations of from 0.01% to 10%. In a more preferred embodiment, low methoxyl pectin with calcium chloride or magnesium chloride from 0.01% to 10% is used in place of agar (see below for examples of pectin solid-phase media for Salmonella detection and quantification). The use of pectin in place of agar saves time and energy in the preparation of the solid-phase media and easier to prepare at room temperature.

Preparation of the 24 Hour Salmonella Agar Kit Vessel I with Solid-Phase Culture Gel Capsule and Liquid-Phase Culture/Enrichment Broth (a Preferred Embodiment)

1. As a solid-phase, a volume of gel agar media is prepared by boiling 0.5% NaCl, 1% peptone, 2% agar, and 1% magnesium chloride in distilled water until agar granules are completely melted.

2. Brilliant Green (12.5 ppm) and phenol red (0.8%) (1% phenol red, sodium salt stock in sterilized water) are then added. The final pH (5.0-6.0) is adjusted by adding HCl into the boiled agar media.

3. Then, the boiled gel agar media is cooled to approximately 50° C.

4. In laboratory studies, 13 mL of gel agar media is placed in a sturdy vessel, such as sterilized polypropylene or glass tube (20 mm O.D.×38 mm length), and cooled at room temperature until solidified.

5. As a liquid phase, sterile buffered peptone water is used as an enrichment broth in plastic or STOMACHER® bags or in another container when ready for sample testing.

6. The Salmonella Agar Kit Vessel I is now prepared for use. The individual assay formulations (including gel agar media) can be stored separately under standard operating procedures and conditions; and then combined with fresh sterile buffered peptone water before use as provided above.

As discussed above, alternative formulations of the media components can be used in other embodiments (i.e., other agar or solid-phase/gelling agent mixtures, selective and non-selective media, chemicals, volume, and concentration, etc.) of the present invention.

Preparation of the 24 Hour Salmonella Pectin Kit Vessel I with Solid-Phase Culture Gel Capsule and Liquid-Phase Culture/Enrichment Broth (a Preferred Embodiment)

1. As a solid-phase, a volume of gel pectin media is prepared by boiling 1% peptone, 1% low methoxyl pectin, 0.5% NaCl, and 1% magnesium chloride in distilled water until pectin is completely dissolved.

2. Brilliant Green (12.5 ppm) and phenol red (0.8%) (1% phenol red, sodium salt stock in sterilized water) are then added. The final pH (5.0-6.0) is adjusted by adding HCl into the gel pectin media.

3. Then, the boiled pectin media is cooled at room temperature.

4. In laboratory studies, 13 mL of pectin media is placed in a sturdy vessel, such as sterilized polypropylene or glass tube (20 mm O.D.×38 mm length), and cooled at room temperature.

5. 2% calcium chloride (50-500 μL) is immediately added to the top pectin media to gel the pectin.

6. As a liquid phase, sterile buffered peptone water is used as an enrichment broth in plastic or STOMACHER® bags or in another container when ready for sample testing.

7. The Salmonella Pectin Kit Vessel I is now prepared for use. The individual assay formulations (including gel pectin media) can be stored separately under standard operating procedures and conditions; and then combined with fresh sterile buffered peptone water before use as provided above.

The alternative formulations of Salmonella media described above for the agar kits may also be used to supplement or modify the pectin kits.

Preparation of the 24-h Salmonella Agar Kit Vessel II with Solid-Phase Culture Gel and Liquid-Phase Culture/Enrichment Broth

1. As a solid-phase culture agar gel, a volume of bottom agar is prepared by boiling 0.5% NaCl, 1% peptone, 2% agar and 1% magnesium chloride in distilled water until agar granules are completely melted.

2. Brilliant Green (12.5 ppm) and phenol red (0.8%) (1% phenol red, sodium salt stock in sterilized water) are added. The final pH (5.0-6.0) is adjusted by adding HCl into the boiled agar media.

3. Then, the agar is cooled to approximately 50° C.

4. In laboratory studies, 7-10 mL of solid-phase culture agar gel is placed in a sterilized culture tube (18 mm O.D.×150 mm length) and cooled at room temperature until solidified.

5. As a liquid-phase culture/enrichment broth, 10-20 mL of sterile buffered peptone water can be placed on the top of the agar gel.

The Salmonella Agar Kit Vessel II is now prepared for use. The individual assay formulations (including gel agar media) can be stored separately under standard operating procedures and conditions; and then combined with fresh sterile buffered peptone water before use as provided above.

As discussed above, alternative formulations of the media components can be used in other embodiments (i.e., other agar or solid-phase/gelling agent mixtures, selective and non-selective media, chemicals, volume, and concentration, etc.) of the present invention.

Preparation of the 24-h Salmonella Pectin Kit Vessel II with Solid-Phase Culture Gel and Liquid-Phase Culture/Enrichment Broth

1. As a solid-phase culture pectin gel, a volume of bottom pectin gel is prepared by boiling 0.5% NaCl, 1% peptone, 1% low methoxyl pectin and 1% magnesium chloride in distilled water until the pectin is completely dissolved.

2. Brilliant Green (12.5 ppm) and phenol red (0.8%) (1% phenol red, sodium salt stock in sterilized water) are added. The final pH (5.0-6.0) is adjusted by adding HCl into the pectin media.

3. Then, the pectin media is cooled at room temperature.

4. 2% calcium chloride (50-500 μL) is immediately added to the top pectin media to gel the pectin.

5. In laboratory studies, 7-10 mL of solid-phase culture pectin gel is placed in a sterilized culture tube (18 mm O.D.×150 mm length) and cooled at room temperature until solidified.

6. As a liquid-phase culture/enrichment broth, 10-20 mL of sterile buffered peptone water can be placed on the top of the pectin gel.

7. The Salmonella Pectin Kit Vessel II is now prepared for use. The individual assay formulations (including gel pectin media) can be stored separately under standard operating procedures and conditions; and then combined with fresh sterile buffered peptone water before use as provided above.

The alternative formulations of Salmonella media described above for the agar kits may also be used to supplement or modify the pectin kits.

Preparation of the 24-h Salmonella Confirmation Agar Kit Vessel III with Solid-Phase Culture Gel and Liquid-Phase Culture/Enrichment Broth

1. As a solid-phase culture gel, a volume of bottom agar is prepared by boiling 0.5% NaCl, 1% peptone, 2% agar, and 1% magnesium chloride in distilled water until agar granules are completely melted.

2. Brilliant Green (12.5 ppm) and phenol red (0.8%) (1% phenol red, sodium salt stock in sterilized water) are added. The final pH (5.0-6.0) is adjusted by adding HCl into the boiled agar media.

3. Then, the agar is cooled to approximately 50° C.

4. In laboratory studies, 7-10 mL of solid-phase culture gel is placed in a sterilized culture tube (18 mm O.D.×150 mm length) and cooled at room temperature until solidified.

5. As a liquid-phase culture/enrichment broth, 10-20 mL of Rappaport-Vassiliadis Salmonella (RVS) Broth, RV R10 broth, or modified RVS broth with reduced protein (i.e., peptone) concentration (0.1-0.4%) and malachite green oxalate or Brilliant Green (0.1-100 ppm) can be placed on the top of the solid-phase culture agar gel. RVS and these similar liquid-phase culture media are not suitable for an initial enriching liquid-phase culture broth because they decrease the sensitivity of the Salmonella assay kits, but are combined with the solid-phase in the assay kits because the combination gives high specificity on Salmonella, which is especially useful as a confirmatory assay.

6. The Salmonella Agar Kit Vessel III is now prepared for use. The individual assay formulations (including gel agar media) can be stored separately under standard operating procedures and conditions; and then combined with fresh sterile buffered peptone water before use as provided above.

As discussed above, alternative formulations of the media components can be used in other embodiments (i.e., other agar or solid-phase/gelling agent mixtures, selective and non-selective media, chemicals, volume, and concentration, etc.) of the present invention.

Preparation of the 24-h Salmonella Confirmation Pectin Kit Vessel III with Solid-Phase Culture Gel and Liquid-Phase Culture/Enrichment Broth

1. As a solid-phase culture gel, a volume of bottom pectin gel is prepared by boiling 0.5% NaCl, 1% peptone, 1% low methoxyl pectin and 1% magnesium chloride in distilled water until the pectin is completely dissolved.

2. Brilliant Green (12.5 ppm) and phenol red (0.8%) (1% phenol red, sodium salt stock in sterilized water) are added. The final pH (5.0-6.0) is adjusted by adding HCl into the pectin media.

3. Then, the pectin gel is cooled at room temperature.

4. In laboratory studies, 7-10 mL of solid-phase culture pectin is placed in a sterilized culture tube (18 mm O.D.×150 mm length) and cooled at room temperature.

5. 2% calcium chloride (50-500 μL) is immediately added to the top pectin media to gel the pectin.

6. As a liquid-phase culture/enrichment broth, 10-20 mL of Rappaport-Vassiliadis Salmonella (RVS) Broth, RV R10 broth, or modified RVS broth with reduced protein concentration (i.e., peptone) concentration (0.1-0.4%) and malachite green oxalate or Brilliant Green (0.1-100 ppm) can be placed on the top of the solid-phase culture pectin gel. RVS and these similar liquid-phase culture media are not suitable for an initial enriching liquid-phase culture broth because they decrease the sensitivity of the Salmonella assay kits, but are combined with the solid-phase in the assay kits because the combination gives high specificity on Salmonella, which is especially useful as a confirmatory assay.

7. The Salmonella Pectin Kit Vessel III is now prepared for use. The individual assay formulations (including gel pectin media) can be stored separately under standard operating procedures and conditions; and then combined with fresh peptone water before use as provided above.

The alternative formulations of Salmonella media described above for the agar kits may also be used to supplement or modify the pectin kits.

Salmonella Assay Kits

The present invention relates to novel Salmonella assay kits comprised of a novel combination of selective ingredients that can be used to detect and quantify the population of Salmonella, including pathogenic Salmonella in preferred embodiments, present in a test sample. The Salmonella assay kits include, but are not limited to, the following: instructions for storage, use, and detection/quantification; assay block/vessel(s); selective and/or non-selective (buffered peptone water) pre-enrichment liquid media; selective solid or semi-solid media; selective liquid media; and color chart to read sample.

The Salmonella assay kits include a singular or a plurality of vessels (which may include test tubes, culture blocks, microculture plates, or other vessel form of appropriate size based on sample screening size or through-put requirements) whereby each vessel contains biochemicals of the detection mechanism in a stable matrix coupled with a growth medium in the same test vessel also in a stable matrix. Preferably, the Salmonella assay kit also includes a specific set of instructions for storage, use, and detection. The Salmonella assay kit may also contain a color chart specific to the signal indicating medium (biochemicals of the detection mechanism) provided. The Salmonella assay kit may be sold on an individual or bulk basis and may be incorporated to include components necessary to perform the steps or processes common to the process of obtaining a viable sample from the environment, industrial setting, water, food sample, or other sample source.

Salmonella Assay Kit Methods

Step 1 (Inoculation/Incubation): Test samples are taken in accordance with normal and well-known laboratory and industrial setting procedures. Test samples of an appropriate dilution(s) are inoculated in an appropriate volume of sterile buffered peptone water (liquid-phase, which can optionally be pre-warmed to 37-42° C.) in a sterile sealable plastic bag or other container. Three to five prepared Salmonella assay kit vessels I are then added to the sample bag/container and incubated at 42° C. for a period of about 24 hours. Alternatively, as discussed above, an appropriate volume of the inoculated liquid-phase media can be added to one or more prepared Salmonella assay kit vessel II and incubated at 42° C. for a period of about 24 hours. For quantification, replicates of serial dilutions are inoculated and incubated in Step 1 following well-known procedures for most probable number analysis (see, e.g., Scott Sutton, The Most Probable Number Method and Its Uses in Enumeration, Qualification, and Validation, J. Valid. Tech. vol. 16, no. 3 (Summer 2010), which is hereby incorporated by reference in its entirety). For example, dilutions can be log base 10, log base 2, or other serial dilution.

Step 2 (Reading): After about 24 hours of incubation, a positive result for Salmonella in a Salmonella assay kit samples show orange or yellow color on the bottom of vessel I or vessel II (bottom gel media) while negative samples stay green on the bottom (based on the biochemical indicators included in the preferred embodiments described above).

Optional Step 3 (Confirmation Inoculation/Incubation): In an optional confirmation stage, positive test samples after Step 2 are used to inoculate an appropriate volume of RVS liquid-phase selective media to one or more prepared Salmonella assay kit vessel III and incubated at 42° C. for a period of about 24 hours.

Optional Step 4 (Confirmation Reading): After about 24 hours of incubation, a positive result for Salmonella in a Salmonella assay kit samples show orange or yellow color on the bottom of vessel III (bottom gel media) while negative samples stay green on the bottom (based on the biochemical indicators included in the preferred embodiments described above).

### Example 1

### Listeria Assay Kit

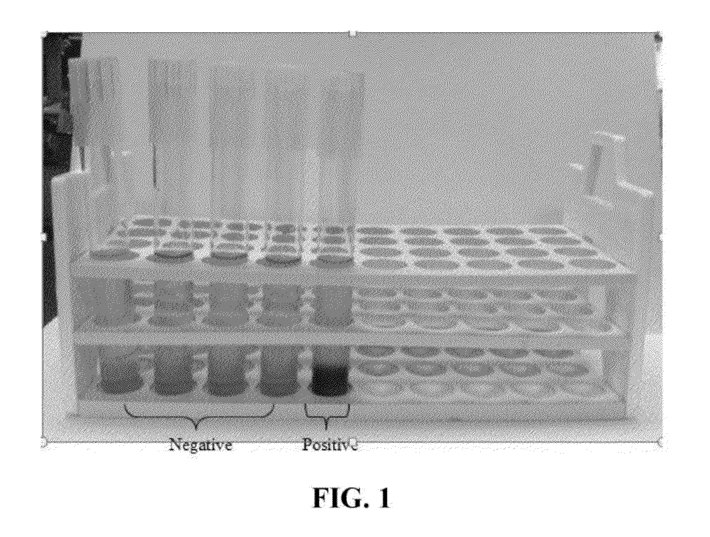

Known positive and negative Listeria samples were used to test a set of Listeria Agar Assay Kit vessels. Five Listeria Agar Assay Kit vessels (1-2 mL of top agar media and 4-10 mL of bottom agar media in sterile test tubes) were prepared as described above in the preferred embodiments. A sufficient volume of Listeria liquid-phase culture/enrichment broth was prepared as described above in the preferred embodiments, and about 10 mL of the liquid-phase media was added to each vessel. The known positive (one total) and negative (four total) Listeria samples were then used to inoculate one each of the five prepared Listeria Agar Assay Kit vessels and then incubated for 24 hours at 37° C. After 24 hours of incubation the vessels were read for detection of Listeria presence in the known samples. As shown in FIG. 1, vessels 1-4 containing the known negative Listeria samples (left side of test tube rack) are all negative for Listeria presence, which can be seen with the yellow (remaining) color of the bottom agar media. Also shown in FIG. 1, vessel 5 containing the known Listeria positive sample (right of vessels 1-4) is positive for Listeria presence, which can be seen with the black (changed) color of the bottom agar media.

### Example 2

### Salmonella Assay Kit

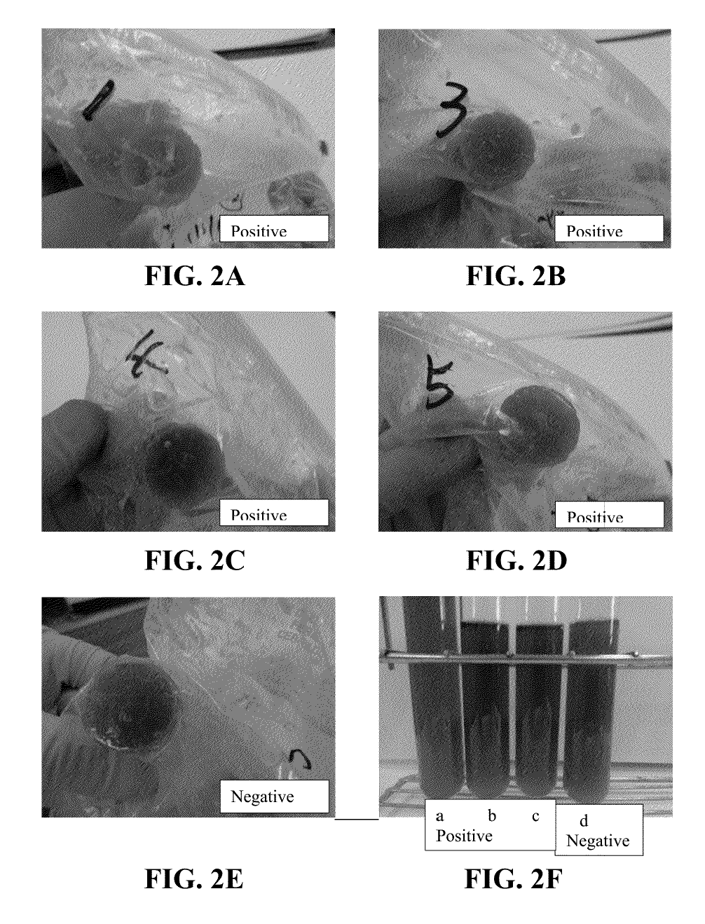

Known positive and negative Salmonella samples were used to test a set of Salmonella Agar Assay Kit vessels (I-III). Five Salmonella Agar Assay Kit vessels I (13 mL of bottom agar media in sterile propylene test tube caps) were prepared as described above in the preferred embodiments. A sufficient volume of Salmonella liquid-phase culture/enrichment broth was prepared as described above in the preferred embodiments, and about 10-5,000 mL of the liquid-phase media (sterile buffered peptone water) was added to each of five large sealable plastic bags. The known positive (four total) and negative (one total) Salmonella samples were then used to inoculate one each of the five prepared bags and 3-5 Salmonella Agar Assay Kit vessels I were added to each bag, then sealed and incubated for 24 hours at 42° C. In parallel, Salmonella Agar Assay Kit vessels II were prepared as described above in the preferred embodiments and 10-20 mL of the liquid-phase media from each of the sealable bags 1-5 were added to the Salmonella Agar Assay Kit vessels II and incubated for 24 hours at 42° C. After 24 hours of incubation the vessels I and II were read for detection of Salmonella presence in the known samples. As shown in FIGS. 2A through 2D, bags 1, 3, 4 and 5 with vessels I containing the known positive Salmonella samples are all positive for Salmonella presence, which can be seen with the orange (changing) color of the bottom agar media in the vessels I. As shown in FIG. 2E, the vessels I in sealable bag 5 containing the known Salmonella negative samples is negative for Salmonella presence, which can be seen with the green (remaining) color of the bottom agar media in the vessels I. The parallel inoculated vessels II samples are shown in FIG. 2F. Tubes a, b, and c all contain known positive Salmonella samples, and each shows positive for Salmonella presence, which can be seen with the yellow and/or orange (changing) color of the bottom agar media in the vessels II. Tube d, however, contains the known negative Salmonella sample, and this vessel II is negative for Salmonella presence, which can be seen with the green to light green (remaining) color of the bottom agar media in the vessel II.

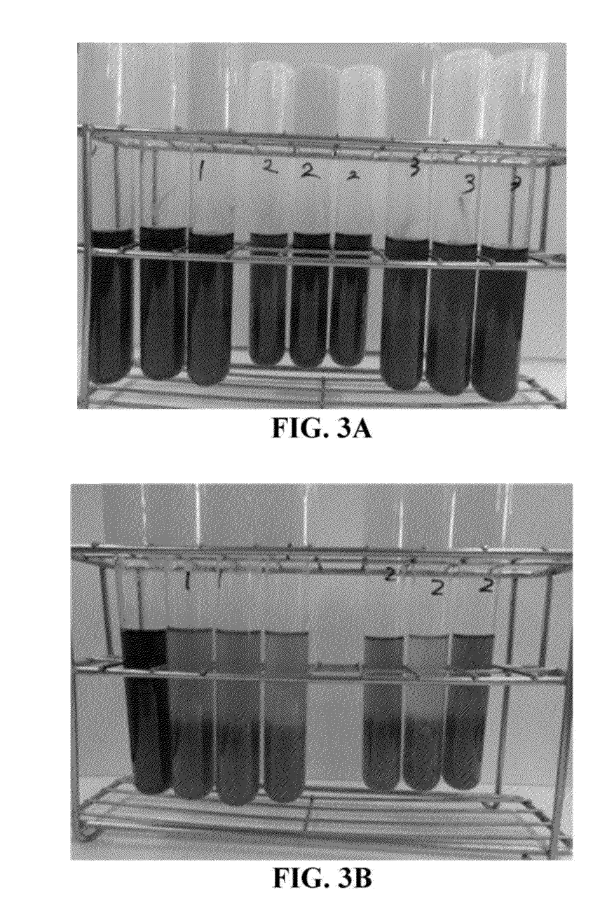

Following the results in vessels I & II above, confirmation tests were performed on the positive and negative 24 hour culture samples in triplicate Salmonella Agar Assay Kit vessels III, which were prepared as described above in the preferred embodiments using RVS broth as liquid-phase media (shown in FIG. 3A prior to inoculation). For inoculation, 0.1 mL of the liquid-phase media from the vessel I sealed bags and liquid-phase media from the vessels II were added to the triplicate sets of vessels III for each sample vessel I bag/vessel II. The inoculated Salmonella Agar Assay Kit vessels III were then incubated for 24 hours at 42° C. After 24 hours of incubation the vessels III were read for confirmation of Salmonella presence in the samples. As shown in FIG. 3B, triplicate vessels III marked “1” and “2” contain inoculant from positive Salmonella samples from either a vessel I sealable bag or a vessel II sample, and each shows positive for Salmonella presence, which can be seen with the orange (changing) color of the bottom agar media and yellowing of the liquid-phase media in the vessels III. The triplicate vessels III marked “3” (one tube shown at left of test tube rack), however, contains inoculant from the negative Salmonella sample from a vessel I or vessel II test, and this vessel III is negative for Salmonella presence, which can be seen with the green to light green (remaining) color of the bottom agar media and liquid-phase media in the vessel III.

The terms “comprising,” “including,” and “having,” as used in the claims and specification herein, shall be considered as indicating an open group that may include other elements not specified. The terms “a,” “an,” and the singular forms of words shall be taken to include the plural form of the same words, such that the terms mean that one or more of something is provided. The term “one” or “single” may be used to indicate that one and only one of something is intended. Similarly, other specific integer values, such as “two,” may be used when a specific number of things is intended. The terms “preferably,” “preferred,” “prefer,” “optionally,” “may,” and similar terms are used to indicate that an item, condition or step being referred to is an optional (not required) feature of the invention.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims. It will be apparent to one of ordinary skill in the art that methods, devices, device elements, materials, procedures, and techniques other than those specifically described herein can be applied to the practice of the invention as broadly disclosed herein without resort to undue experimentation. For example, such modifications as additional or different color-indicating interactions, materials, chemicals, gradient layers, concentration of materials, pH of all solid, semi-solid and liquid media, size/volume of vessels such as microplates and material of vessels could be used. All art-known functional equivalents of methods, devices, device elements, materials, procedures, and techniques described herein are intended to be encompassed by this invention. Whenever a range is disclosed, all subranges and individual values are intended to be encompassed. This invention is not to be limited by the embodiments disclosed, including any exemplified in the specification, which are given by way of example and not of limitation.

All references throughout this application, for example patent documents including issued or granted patents or equivalents, patent application publications, and non-patent literature documents or other source material, are hereby incorporated by reference herein in their entireties, as though individually incorporated by reference, to the extent each reference is at least partially not inconsistent with the disclosure in the present application (for example, a reference that is partially inconsistent is incorporated by reference except for the partially inconsistent portion of the reference).

1. A Listeria detection assay vessel for detecting the presence of a Listeria bacteria comprising: at least one solid-phase gel media and a signal indicator disposed in a single culture vessel; and a liquid-phase media, wherein the at least one solid-phase gel media reduces Type I errors in detecting Listeria bacteria by comprising at least one component that suppresses growth of non-target bacteria and wherein the liquid-phase media reduces Type II errors in detecting Listeria bacteria by comprising at least one component that enhances growth of target bacteria.

2. The Listeria detection assay vessel of claim 1, wherein the at least one solid-phase gel media comprises esculin.

3. The Listeria detection assay vessel of claim 2, wherein the signal indicator is ferric ammonium citrate.

4. The Listeria detection assay vessel of claim 1, wherein the at least one solid-phase gel media comprises agar.

5. The Listeria detection assay vessel of claim 1, wherein the at least one solid-phase gel media comprises pectin.

6. The Listeria detection assay vessel of claim 1, wherein the at least one solid-phase gel media comprises a bottom solid-phase gel media and a top solid-phase gel media.

7. The Listeria detection assay vessel of claim 1, wherein the liquid-phase media comprises one or more of the following: pancreatic digest of casein, proteose peptone #3, beef extract, yeast extract sodium chloride, disodium phosphate, monopotassium phosphate, and esculin.

8. The Listeria detection assay vessel of claim 1, wherein the liquid-phase media comprises nalidixic acid, acriflavine HCl, antibiotics, or combinations thereof.

9. The Listeria detection assay vessel of claim 1, wherein the liquid-phase media is added to the Listeria detection assay vessel after being inoculated with a test sample.

10. A method of detecting a Listeria bacteria comprising:
(a) inoculating the liquid-phase media of at least one Listeria detection assay vessel of claim 1 with a sample suspected of harboring Listeria bacteria;
(b) incubating the inoculated vessel of step (a) for about 24 hours at 35-37° C.; and
(c) reading the incubated vessel of step (b) for a signal indication for a presence or an absence of Listeria bacteria.

11. The method of claim 10, further comprising inoculating a liquid-phase media for enrichment of Listeria bacteria with the sample suspected of harboring Listeria bacteria and incubating the inoculated liquid-phase media for enrichment of Listeria bacteria prior to step (a).

12. The method of claim 11, wherein the incubated liquid-phase media for enrichment of Listeria bacteria is used in lieu of the liquid-phase media in step (a).

13. The method of claim 10, wherein the Listeria bacteria detected is Listeria welshimeri, Listeria innocua, Listeria ivanovii, Listeria grayi, Listeria seeligeri, Listeria monocytogenes, or combinations thereof.

14. A kit for detecting the presence of Listeria bacteria comprising at least one Listeria detection assay vessel and instructions; wherein the at least one Listeria detection assay vessel comprises at least one solid-phase gel media and a signal indicator disposed in a single culture vessel; and a liquid-phase media, wherein the at least one solid-phase gel media reduces Type I errors in detecting Listeria bacteria by comprising at least one component that suppresses growth of non-target bacteria and wherein the liquid-phase media reduces Type II errors in detecting Listeria bacteria by comprising at least one component that enhances growth of target bacteria.

15. The kit of claim 14, wherein the at least one Listeria detection assay vessel is prepackaged with prepared media for the at least one solid-phase gel media and the liquid-phase media.

16. The kit of claim 14, wherein the at least one Listeria detection assay vessel is prepackaged as components for a user to prepare media for the at least one solid-phase gel media and the liquid-phase media.

17. The kit of claim 15, wherein the at least one Listeria detection assay vessel is prepackaged with prepared media for the at least one solid-phase gel media and the liquid-phase media within the single culture vessel.

18. The kit of claim 14, further comprising enrichment culture vessels and Listeria enrichment liquid-phase media.

19. The kit of claim 16, wherein the instructions comprise directions for a user to prepare the at least one Listeria detection assay vessel.

20. The kit of claim 14, wherein the at least one solid-phase gel media comprises a bottom solid-phase gel media and a top solid-phase gel media, and wherein the bottom solid-phase gel media comprises esculin.

21. The kit of claim 20, wherein the bottom solid-phase gel media comprises the signal indicator which is ferric ammonium citrate.

22. The kit of claim 14, wherein the at least one solid-phase gel media comprises agar.

23. The kit of claim 14, wherein the at least one solid-phase gel media comprises pectin.

24. The kit of claim 14, wherein the liquid-phase media comprises one or more of the following: pancreatic digest of casein, proteose peptone #3, beef extract, yeast extract sodium chloride, disodium phosphate, monopotassium phosphate, and esculin.

25. The kit of claim 14, wherein the liquid-phase media comprises nalidixic acid, acriflavine HCl, antibiotics, or combinations thereof.

26. The kit of claim 14, wherein the liquid-phase media is added to the Listeria detection assay vessel after being inoculated with a test sample.

27. A method of detecting a Salmonella bacteria comprising:
inoculating a first liquid-phase media in a culture container with a sample suspected of harboring Salmonella bacteria;
placing in the culture container at least one first Salmonella detection assay vessel containing a first solid-phase gel media and a first signal indicator;
incubating the at least one first Salmonella detection assay vessel for about 24 hours at about 42° C.; and
reading the at least one first Salmonella detection assay vessel after incubation for a signal indication for a presence or an absence of Salmonella bacteria.

28. The method of claim 27, wherein the first liquid-phase media is disposed in an at least one second Salmonella detection assay vessel containing a second solid-phase gel media and a second signal indicator and the placing step is omitted.

29. The method of claim 28, further comprising after the reading step:
inoculating a second liquid-phase media disposed in at least one third Salmonella detection assay vessel containing a third solid-phase gel media and a third signal indicator with a portion of the first liquid-phase media after the incubating step;
incubating the at least one third Salmonella detection assay vessel for about 24 hours at about 42° C.; and
reading the at least one third Salmonella detection assay vessel after incubation for a confirmatory signal indication for the presence or the absence of Salmonella bacteria.

30. The method of claim 29, wherein the first and second liquid-phase media are different liquid-phase media; the first, second, and third solid-phase gel media are different solid-phase gel media; and the first, second, and third signal indicators are different.

31. The method of claim 27, further comprising after the reading step:
inoculating a second liquid-phase media disposed in at least one third Salmonella detection assay vessel containing a second solid-phase gel media and a second signal indicator with a portion of the first liquid-phase media after the incubating step;
incubating the at least one second Salmonella detection assay vessel for about 24 hours at about 42° C.; and
reading the at least one second Salmonella detection assay vessel after incubation for a confirmatory signal indication for the presence or the absence of Salmonella bacteria.

32. The method of claim 31, wherein the first and second liquid-phase media are different liquid-phase media; the first and second solid-phase gel media are different solid-phase gel media; and the first and second signal indicators are different.

33. A kit for detecting the presence of Salmonella bacteria comprising at least one first Salmonella detection assay vessel, a first liquid-phase media, a first culture container; at least one second Salmonella detection assay vessel, at least a third Salmonella detection assay vessel, and instructions;
wherein the at least one first Salmonella detection assay vessel comprises a first solid-phase gel media and a first signal indicator;
wherein the at least one second Salmonella detection assay vessel comprises a second solid-phase gel media, a second liquid-phase media, and a second signal indicator;
wherein the at least one third Salmonella detection assay vessel comprises a third solid-phase gel media, a third liquid-phase media, and a third signal indicator; and
wherein the first, second, and third solid-phase gel media reduces Type I errors in detecting Salmonella bacteria by comprising at least one component that suppresses growth of non-target bacteria, and wherein the first, second, and third liquid-phase media reduces Type II errors in detecting Salmonella bacteria by comprising at least one component that enhances growth of target bacteria.

34. The kit of claim 33, wherein the at least one first, second, and third Salmonella detection assay vessels are prepackaged with prepared media for the first, second, and third solid-phase gel media and the second and third liquid-phase media.

35. The kit of claim 33, wherein the at least one first, second, and third Salmonella detection assay vessels are prepackaged as components for a user to prepare media for the first, second, and third solid-phase gel media and first, second, and third liquid-phase media.

36. The kit of claim 35, wherein the instructions comprise directions for a user to prepare the at least one first, second, and third Salmonella detection assay vessels.

37. The kit of claim 33, wherein the first, second, and third signal indicators are phenol red.

38. The kit of claim 33, wherein the first, second, and third solid-phase gel media comprises at least one of brilliant green, malachite green oxalate, crystal violet, or antibiotics to suppress non-target bacteria.

39. The kit of claim 33, wherein the first, second, and third solid-phase gel media comprise agar, gelatin, pectin, or combinations thereof.

40. The kit of claim 33, wherein the first, second, and third solid-phase gel media comprise pectin.
